(19) United States
(12) Patent Application Publication
(10) Pub. No.: US 2012/0165576 A1
(43) Pub. Date: Jun. 28, 2012

(54) USE OF ERYTHRITOL FOR THE PREVENTION OR TREATMENT OF HYPERTENSION

(75) Inventors: Aalt Bast, Nijkerk (NL); Alvin Berger, Long Lake, MN (US); Petrus Wilhelmus Hubertus Antonius De Cock, Keerbergen (BE); Gerardus Johannes Martinus Den Hartog, Maastricht (NL)

(73) Assignee: CARGILL, INCORPORATED, Wayzata, MN (US)

(21) Appl. No.: 13/387,129

(22) PCT Filed: Jul. 26, 2010
(86) PCT No.: PCT/US10/43216

§ 371 Date: Mar. 5, 2012

(30) Foreign Application Priority Data
Jul. 27, 2009 (US) 61228785
Jul. 27, 2009 (US) 61228803

(51) Int. Cl. C07C 31/24

(52) U.S. Cl. 568/853

(57) ABSTRACT

The present invention relates to the use of erythritol for the up regulation of the SOD2 enzyme in mammals and prevention or treatment of hypertension. More specifically, erythritol protects against the development of endothelial dysfunction and related reduction of vasodilatation and erythritol protects against hemolysis-induced vasoconstriction.

### CROSS REFERENCE TO RELATED APPLICATION

This application claims the benefit of the U.S. Provisional Patent Application Ser. No. 61/228,803, filed Jul. 27, 2009, and entitled ERYTHRITOL IS A SWEET ANTIOXIDANT and Ser. No. 61/228,785, filed Jul. 27, 2009, and entitled USE OF ERYTHRITOL FOR THE PREVENTION OR TREATMENT OF HYPERTENSION, which is hereby incorporated by reference in its entirety.

### FIELD OF THE INVENTION

The present invention relates to the use of erythritol for the up regulation of the SOD2 enzyme in mammals and prevention or treatment of hypertension. More specifically, erythritol protects against the development of endothelial dysfunction and related reduction of vasodilation and erythritol protects against hemolysis-induced vasoconstriction.

### BACKGROUND OF THE INVENTION

Hypertension or high blood pressure is a condition in which the blood pressure in the arteries is chronically elevated. With every heart beat, the heart pumps blood through the arteries to the rest of the body. Blood pressure is the force of blood that is pushing up against the walls of the blood vessels. If the pressure is too high, the heart has to work harder to pump, and this could lead to organ damage and several illnesses such as heart attack, stroke, heart failure, aneurysm, or renal failure.

Factors that are associated with the condition of hypertension include, but are not limited to, smoking, obesity or being overweight, diabetes, sedentary lifestyle, lack of physical activity, metabolic syndrome, high levels of salt intake, insufficient calcium, potassium, and magnesium consumption, vitamin D deficiency, high levels of alcohol consumption, stress, aging, drugs including oral anticontraceptives, genetics and a family history of hypertension, chronic kidney disease, adrenal and thyroid disorders or certain forms of cancer. Many of these factors involve one or both of the following two mechanisms that cause an increase of blood pressure: (1) the development of endothelial dysfunction, and (2) hemolysis of red blood cells.

Endothelial dysfunction (ED) is the earliest clinically detectable stage of cardiovascular disease characterized by a shift of the actions of the endothelium toward reduced vasodilation, a proinflammatory state, and prothrombic properties. ED contributes to hypertension, coronary artery disease, chronic heart failure, peripheral artery disease, atherosclerosis, diabetes, metabolic syndrome, obesity, inflammation, and chronic renal failure.

The entire circulatory tree is lined by a single epithelial-like layer of vascular endothelial cells (ECs) (Pober et al., 2009). Although ECs exhibit characteristics that vary with anatomic location, both by organ system and by vessel type (e.g., artery, arteriole, capillary, venule, vein), all ECs share common features that distinguish them from other cell types and allow them to perform critical homeostatic functions. Key homeostatic functions include keeping blood fluid, regulating blood flow, regulating macromolecule and fluid exchange with the tissues, preventing leukocyte activation, and aiding in immune surveillance for pathogens. Injury or cell death impairs or prevents conduct of these activities, resulting in dysfunction. Most endothelial cell death is apoptotic, involving activation of caspases, but nonapoptotic death responses also have been described. Stimuli that can cause endothelial injury or death include environmental stresses such as oxidative stress, endoplasmic reticulum stress, metabolic stress, and genotoxic stress, as well as pathways of injury mediated by the innate and adaptive immune systems.

Hemolysis is a component of several diseases such as hypertension, intravascular hemolysis seen in anemia, nicroangiopathic hemolytic anemia, sickle cell disease, paroxysmal nocturnal hemoglobinuria, thalassemias, and hereditary spherocytosis. Hemolysis-associated hypertension can be caused through several mechanisms. The main and immediate is caused by vasoconstriction through the release of red blood cell content into plasma. Further, the release of specifically soluble hemoglobin and arginase into plasma leads to nitric oxide deficiency and a state of endothelial dysfunction that is associated with clinical development of hypertension. Another mechanism relates to the release of adenosine deaminase from injured erythrocytes into plasma and metabolic conversion of adenosine to inosine by adenosine deaminase thereby reducing extracellular adenosine levels. Adenosine, mainly via activation of adenosine A2A receptors, mediates a number of biological responses that reduce hemolysis-induced vasculopathy and the risk of hypertension. Vascular protective effects of adenosine are severely diminished by adenosine deaminase released from injured red blood cells.

Any protection against endothelial dysfunction or hemolysis of red blood cells will assist in reducing the risk of developing hypertension or the treatment thereof.

### SUMMARY OF THE INVENTION

The present invention revolves around the novel and unexpected finding that SOD2 expression in a cell is dramatically up regulated through exposure to erythritol. Erythritol activates the cell's own antioxidant machinery. This activation or up regulation provides a protective effect to the endothelium and prevents endothelial dysfunction and hemolysis. The causal link between endothelial dysfunction and hemolysis has been well documented in the art. Accordingly, this SOD2 up regulating effect of erythritol can be utilized as means to treat or prevent hypertension.

The present invention pertains to the use of erythritol for the manufacture of a product or composition that stimulates expression of SOD2 in an animal or human.

In another preferred embodiment of the present invention, the disease to be prevented or treated is endothelial dysfunction and endothelial dysfunction-induced hypertension.

In another preferred embodiment of the present invention, the disease to be prevented or treated is red blood cell hemolysis and hemolysis-induced hypertension.

In a particularly preferred embodiment, the present invention aims at the prevention or treatment of hypertension in an animal or human.

The present invention includes a method of treating hypertension by administering to an animal or human an effective amount of erythritol. The present invention also includes a method of preventing or treating endothelial dysfunction by administering to an animal or human an effective amount of erythritol. Thirdly, the present invention also includes a method of treating the complications of hyperglycemia by administering to an animal or human an effective amount of erythritol.

In another preferred embodiment, the above-mentioned product or composition is orally administered.

Furthermore, the product or composition may be formulated as a pharmaceutical product or composition, such as in the form of a tablet, capsule, suspension, solution, powder or emulsion. In particular, the product or composition may be formulated in the form of a beverage or food supplemented with erythritol as a hypertension-protective agent.

### BRIEF DESCRIPTION OF THE DRAWINGS

FIG. 1 shows carbachol concentration-response curves of normoglycemic rats (closed circles), normoglycemic rats that had consumed erythritol (1 g/kg/day, open circles), and diabetic rats (triangles).

FIG. 2 shows carbachol concentration-response curves of diabetic rats (closed circle) and diabetic rats that had consumed erythritol (1 g/kg/day, open circles).

FIG. 3 shows the effect of erythritol (0-50 mM) on AAPH-induced hemolysis. Datapoints are average of three separate experiments. Curves are fitted (3 parameter sigmoidal). Erythritol concentration-dependently shifted the curve to the right.

FIG. 4 shows the effect that erythritol has on endothelial cells which results in an increase in manganese superoxide dismutase (SOD2) transcription. Cultured human umbilical vein endothelial cells (CRL-1730, which are available from ATCC) were incubated under standard conditions with erythritol (5 mM, 24 hours). Exposure to erythritol induced or stimulated a 9.4 fold increase in the expression of SOD2 as determined by qPCR.

### DETAILED DESCRIPTION OF THE INVENTION

The present invention is based on the novel observations that (1) the administration of erythritol significantly increases the expression of manganese superoxide dismutase (SOD2), a critical antioxidant enzyme; and (2) erythritol is capable of delaying and reducing hemolysis of red blood cells. Both these findings offer a therapeutic strategy for the long term prevention or treatment of hypertension and the complications or diseases associated therewith.

SOD2 is an antioxidant enzyme which is confined to the mitochondria, but can be released into the extracellular space. SOD2, like the other SODs, which are found in the cytosol (SOD1) and extracellularly (SOD3) converts superoxide radicals into hydrogen peroxide and oxygen, thereby limiting the toxic effects of superoxide radicals. These radicals can cause lipid peroxidation and react with nitric oxide to form the reactive peroxynitrite thus turning the vasorelaxant nitric oxide into the vasotoxic peroxynitrite. The critical role of superoxide radicals in and around endothelial cells in the pathogenesis of hypertension has been recognized for almost 20 years. Expression and activity of SOD2 is known to be reduced in pulmonary artery hypertension.

Vascular oxidative stress has also been identified as the common link between diabetes and hypertension. Our discovery that erythritol stimulates the expression of SOD2 in endothelial cells together with a large amount of previous observations on the role of superoxide radicals in the (pathogenesis of) hypertension as well as the antihypertensive actions of superoxide dismutases clearly indicates that erythritol can be used in the prevention and the attenuation of hypertension and prevention of hemoylsis. Accordingly, the present invention relates to the use of erythritol for the manufacture of a product or composition for the prevention or treatment of endothelial dysfunction and hypertension.

The upregulation of the SOD2 enzyme thus restores the impaired or attenuated endothelium-dependent relaxation of vascular smooth muscle of diabetic rats to essentially the same level as found in normoglycemic rats, and erythritol is capable of delaying and reducing hemolysis of red blood cells. Accordingly, the increased expression of SOD2 will help to maintain normal endothelial function, vascular tone, and normal blood pressure.

Erythritol is a polyol (1,2,3,4-butanetetrol) which is present in small quantities in melons and peaches and currently produced in large quantities for use as a non-caloric, tooth friendly bulk sweetener in confectionery, chewing gum, beverages and bakery products. Erythritol has no impact on blood insulin or glucose levels, which renders it a useful and safe food ingredient for zo patients with hyperglycemia, in particular diabetic patients. Further, erythritol is rapidly and virtually completely absorbed from the gut and is capable of passing through the membranes of the body, which makes it easy to reach appropriate systemic concentration required to exercise its hypertension-protective effects described herein. Additionally, extensive clinical testing has shown that erythritol is well tolerated and has no digestive side-effects, even after consumption of large quantities. For example, bolus doses of up to 50 grams and daily doses up to 80 grams of erythritol have been shown to be well tolerated.

Within the context of the present invention, the term “disease” is intended to mean any deviation from or interruption of the normal structure or function of any part, organ, or system or combination thereof of an animal or human, that is manifested by a characteristic set of symptoms and signs and whose etiology, pathology, and prognosis may be known or unknown. The term “animal” used herein includes mammals, except humans, such as a member of the equine, porcine, bovine, murine, canine or feline species.

According to the present invention, the term “hyperglycemia” refers to a condition in which an excessive amount of glucose is present in the blood serum. The physiological blood glucose level is about 5 to 7 mmol/l. A blood sugar level of 10 mmol/l (or chronically above 7 mmol/l) or more is considered hyperglycemic. Abnormally high blood glucose levels are attributed to a variety of causes including, but not limited to, diabetes, in particular type I or II diabetes, hyperadrenalism, hyperpituarism, paraneoplastic syndrome, treatment with certain medicaments, such as beta blockers, thiazide diuretics, corticosteroids, niacin, pentamidine, certain protease inhibitors, L-asparaginase, several antipsychotic agents, pancreatic diseases, myocardial infarction, stroke or other neurological diseases, renal insufficiency, hepatic insufficiency, acromegaly, Cushing's syndrome, pheochromocytoma, hyperthyroidism, glucagonoma, amyloidosis.

The term “prevention” used within the present invention includes (i) any activity which avoids the development of a disease of an animal or human which may be predisposed to the disease but has not yet been diagnosed as having it, and (ii) any activity which is aimed at early disease detection, thereby increasing opportunities for intervention to prevent progression of the disease and emergence of symptoms. Further, the term “treatment” as used herein covers (i) any activity which protects against a disease by inhibiting the disease, i.e. arrests the disease development, and (ii) any activity which relieves the disease, i.e. causes regression or disappearance of the disease.

The subject in need of the prevention or treatment is a mammalian subject, i.e. humans or any other animal classified as a mammal, including domestic and farm animals, and zoo, sports, or pet animals, such as dogs, cats, cattle, sheep, pigs, goats, rabbits, etc. Preferably, the subject in need of prevention or treatment is a human.

In a preferred embodiment of the present invention, the disease to be prevented or treated is endothelial dysfunction and endothelial dysfunction-induced hypertension.

In another preferred embodiment of the present invention, the disease to be prevented or treated is red blood cell hemolysis or hemolysis-induced hypertension.

In accordance with the present invention, the term “endothelial dysfunction” refers to a condition which is generally characterized by an impairment, attenuation or even loss of smooth muscle vasodilation or relaxation mediated by the endothelium. “Endothelium” means a layer of endothelial cells that line the interior surface of vessels, in particular blood vessels, and constitute an interface between the circulating blood in the lumen and the vessel wall. Thus, the term endothelial dysfunction refers in other words to a reduced endothelium-dependent vasodilator capacity. According to a preferred meaning of the term “endothelial dysfunction”, a reduced endothelium-dependent vasodilator capacity of the coronary and peripheral circulation to acetylcholine or other M₃ receptor agonists is meant.

In a particularly preferred embodiment, the present invention relates to the prevention or treatment of hypertension, wherein the hypertension is associated with diabetes. The expression “associated with diabetes” is in particular meant to include “caused by diabetes”. Erythritol is particularly useful in the prevention or treatment of hypertension of an animal or human. According to the present invention, the product or composition can be administered by various routes. The route of administration is not particularly limited, and is determined by the preparation form, and the condition of the animal or human to be prevented or treated, such as age, sex and the degree of disease. For example, the product or composition can be administered orally, parenterally, subcutaneously, intravenously, intramuscularly, by inhalation spray, topically, rectally, nasally, buccally, vaginally, or transdermally. Since erythritol is rapidly and virtually completely absorbed from the gut, the preferred route of administration is orally.

The product or composition of the present invention is most preferably formulated in the form of a beverage, food or any other orally used product targeted at a subpopulation of humans or animals that suffer from chronically elevated blood glucose levels. The term “beverage” used herein refers to drinks specifically prepared for animal or human consumption which consist largely of water. Examples of beverages for use within the present invention include existing or newly developed soft drinks, dairy drinks, juice-based drinks and mixtures thereof. The term “food” used herein includes any existing or newly developed foodstuff, except beverages, intended to be ingested by animals or humans.

Erythritol has the advantage that it can be easily incorporated into food, beverages or other orally used products, such as by simple mixing a particular existing or newly developed drink or food with erythritol. As mentioned herein above, erythritol is particularly suited for incorporation in beverages and food since it is rapidly and virtually completely absorbed from the gut, is well tolerated and has no digestive side-effects, even after consumption of large quantities. In addition, erythritol is not systemically metabolized and can therefore continue to exercise its in vivo beneficial effects that are the subject of this patent application. Excretion of erythritol with the urine is complete about 3 days after it has been administered.

The product or composition of the invention may also be formulated in the form of a pharmaceutical product or composition for human or animal consumption. The term “pharmaceutical product or composition”, as used herein, refers to a chemical product or composition capable of inducing a desired therapeutic effect, when properly administered to a patient, and which is formulated by mixing erythritol and optionally other active ingredients with one or more well-known substances selected from physiologically acceptable carriers, diluents, and other agents that are usually incorporated into pharmaceutical formulations to provide improved transfer, delivery, tolerance, and the like. In addition, compounds such as antioxidants, dispersants, emulsifiers, flavorings, sweeting agents, coloring agents and preservatives may also be included in the pharmaceutical product or composition.

Suitable forms of the pharmaceutical product or composition of the present invention include, for example, solid forms, such as powders, tablets, pills, capsules, cachets, suppositories and granules, and liquid forms, such as solutions, syrups, suspensions and emulsions. The appropriate form of the pharmaceutical product or composition is primarily guided by the route of administration, the desired release profile, and other factors such as incompatibilities of active zo substance and pharmaceutical excipients. A person skilled in the art of pharmaceutical formulations is able to choose in routine fashion the form and preparation method with reference to known material and process parameters. For oral administration, for example, tablets may contain carriers, such as, but not limited to, polyols, lactose and corn starch, and/or lubricating agents, such as magnesium stearate. Further, capsules may contain diluents including, but not limited to, polyols, lactose and dried corn starch. Moreover, aqueous solutions or suspensions may contain emulsifying and suspending agents.

Erythritol is known to be well tolerated in single doses of up to approximately 50 grams and daily doses up to 80 grams. Because of this level of tolerance, the positive effects of erythritol can be expected to be seen over a wide range of daily doses such as from 1 mg to 2500 mg per kilogram body weight of an animal or human. Preferred doses include from 2 mg to 1000 mg per kilogram of body weight. Preferably, the erythritol-containing products or compositions of the present invention are in unit dosage form. In such form the product or composition is subdivided into unit doses containing appropriate quantities of erythritol. The quantity of erythritol in a unit dose may vary from 10 mg to 50,000 mg, preferably from 1,000 mg to 25,000 mg. Doses of this level could be easy to achieve through oral consumption of typical foods, beverages, or confectionary products. For example, 200 grams of a yogurt containing 10% erythritol would easily provide a 20 gram dose. Small hard candies or tablets can be prepared containing erythritol and range from 0.5 to 5 grams each. Beverages can typically include 0.5% to 20% erythritol and would conveniently provide single doses of between 1 and 40 grams.

The specific daily dose for a particular animal or human will vary depending upon a variety of factors, including the age, body weight, general health, sex and diet of the animal or human, and form and time of administration. The considerations for determining the proper dosage levels are well-known to a person skilled in the art.

According to the present invention, any administration regimen regulating the timing and sequence of administration can be used and repeated as necessary to effect prevention or treatment of hypertension and the diseases caused thereby. Further, the erythritol-containing is product or composition of the present invention may be co-administered with one or more additional active agents. Alternatively or additionally, the product or composition of the present invention may contain one or more further active agents.

The invention will now be illustrated by way of an example, which is not to be construed as imposing limitations on the invention.

### Example 1

In the following, the inventors of the present invention demonstrate the use of erythritol for the prevention or treatment of endothelial dysfunction-induced hypertension by in vivo experiments. In these in vivo experiments, the endothelial-dependent smooth muscle relaxation of normally glycemic or diabetic rats which received normal drinking water or water supplemented with erythritol were studied. More specifically, the effect of erythritol on the endothelial function was determined in organ baths using sections of thoracic aorta (aortic rings). Contraction was elicited by stimulating with phenylephrine (PE), an α-adrenergic receptor agonist. Subsequently, endothelium-dependent relaxation was analyzed by determining concentration-response curves with the muscarinic receptor agonist carbachol (CAR). Finally, maximum relaxation was induced by the addition of sodium nitroprusside (SNP), a compound that releases NO and thus bypasses the endothelial NO production.

It was found that the endothelial-dependent relaxation in response to the vasorelaxant carbachol was markedly attenuated in diabetic rats. The administration of erythritol resulted in a relaxation response to CAR which is essentially the same as the relaxation response of normoglycemic rats.

Thus, the conducted in vivo experiments, which are further explained in the sections that follow, represent the identification of a novel therapeutic strategy for the prevention or treatment of hypertension by administering erythritol in a suitable mode of administration and amount to animals or humans suffering from chronically elevated blood glucose levels, such as animals or humans afflicted with diabetes.

### Experimental Procedures

20 Wistar rats (10 males, 10 females, 200 to 250 g body weight) were used in these in vivo studies which were performed with the approval of the Ethics Committee for animals of the University of Maastricht. The rats were supplied by Charles River Nederland BV and kept under standard conditions, wherein standard food (Ssniff Spezialdiäten GmbH, Soest, Germany) and acidified drinking were provided ad libitum. Diabetes mellitus was induced according to the protocol of Hasselbaink et al., according to which the rats were anesthetized with halothane and then treated with 70 mg/kg streptozotocin (STZ) in citrate buffer (100 mM, pH 4.5) via i.v. injection in the tail vein. Controls received citrate buffer only. After 7 days, blood glucose was determined with a glucose meter (Lifescan BV, Maastricht, Netherlands).

The thus obtained 10 diabetic rats were divided in two groups: 1) diabetic rats, receiving normal drinking water (group D, N=5), and 2) diabetic rats, receiving erythritol-supplemented water (group DE, N=5) for 21 days. The 10 non-diabetic rats were also divided in two groups: 1) normoglycemic rats, receiving normal drinking water (group N,N=5), and 2) normoglycemic rats, receiving erythritol-supplemented water (group NE, N=5) for 21 days. The amount of solution drunk by each individual rat was monitored and the concentration of the erythritol solution was readjusted daily to assure that each group NE and DE rat had a daily consumption of 1 g erythritol per kg body weight. The other groups (N and D) were given normal drinking water ad libitum. Weight and blood glucose levels of the rats were determined weekly.

After 3 weeks, the rats were sacrificed with CO₂/O₂ and blood was rapidly collected by venipuncture. Then the thoracic parts of the aortas were excised and immediately placed in Krebs-Ringer buffer (118 mM NaCl, 4.4 mM KCl, 2.5 mM CaCl₂, 1.2 mM KH₂PO₄, 1.2 mM MgSO₄, 10 mM glucose, and 25 mM NaHCO₃) after which excess fat and connective tissue were removed. Rings of approximately 4 mm in length were cut and mounted between stainless steel hooks in a 20 ml organ bath filled with Krebs-Ringer buffer, kept at 37° C. and bubbled with 95% O₂-5% CO₂.

An isometric force transducer (Grass FT03) was attached to each strip after which the resting tension was constantly readjusted to 1.0 g for 60 min. Integrity of the tissues was checked with phenylephrine (PE; 10 μM) and carbachol (CAR; 100 μM), followed by a washout period of 30 minutes. The aortic rings were then submaximally contracted with phenylephrine (10 μM). When a stable contraction was established a carbachol concentration-response curve (10 nM to 100 μM) was recorded. When a stable relaxation was obtained after the last addition of carbachol, sodium nitroprusside (SNP, 100 μM) was added to the organ bath to induce maximum relaxation.

### Results

The blood glucose levels of the streptozotocin (STZ) diabetic rats measured 7 days after the STZ treatment were increased to 25.0±1.4 mM compared to 6.7±0.2 mM of the control rats. The endothelial function of the 4 different experimental groups (N, NE, D, and DE) was determined by measuring the response of isolated aortic rings to phenylephrine (PE), carbachol (CAR) and sodium nitroprusside (SNP). The results are shown in Table 1.

TABLE 1
Aorta reactivity measurements

| Experi- | | | | | SNP |
|---|---|---|---|---|---|
| mental | PE | CAR | SNP | Tot. relax | fraction |
| group | (g) | (g) | (g) | (g) | (%) |
| N | 0.66 ± 0.09 | 0.63 ± 0.07 | 0.13 ± 0.03 | 0.76 ± 0.09 | 17.4 ± 3.1 |
| NE | 0.70 ± 0.06 | 0.60 ± 0.07 | 0.21 ± 0.06 | 0.81 ± 0.09 | 24.7 ± 5.5 |
| D | 0.62 ± 0.02 | 0.48 ± 0.03 | 0.30^{a} ± 0.07 | 0.78 ± 0.04 | 36.8 ± 7.0^{a} |
| DE | 0.60 ± 0.07 | 0.61 ± 0.07 | 0.14 ± 0.03 | 0.75 ± 0.09 | 17.8 ± 3.1^{b} |

Values are means ± SEM, N = 5 for groups N and NE; N = 4 for groups D and DE
PE: Contraction induced by 10 μM phenylephrine
CAR: relaxation induced by 100 μM carbachol
SNP: Relaxation induced by 100 μM SNP after stable response to 100 μM carbachol was obtained
Tot. relax. Relaxation induced by 100 μM carbachol followed by 100 μM SNP
SNP fraction: Contribution of 100 μM SNP to the total relaxation.
^{a}p < 0.05 compared to N
^{b}p < 0.05 compared to D

As apparent from Table 1, 10 μM phenylephrine caused an identical contraction in the aortic rings from all four groups. However, the response to the vasorelaxant compound carbachol was markedly attenuated in the diabetic rats (D group). Total relaxation of the aortic rings from the diabetic rats was not smaller compared to other groups, but had to be provoked for a larger part by the NO donating compound SNP. This indicates that the relaxant capacity of the vascular zo smooth muscle is not affected in the diabetic rats. The attenuated response to carbachol in diabetic rats combined with the unaltered total relaxation provides strong evidence that the endothelium of the diabetic rats was damaged and therefore not capable of generating sufficient NO to induce maximum relaxation.

Surprisingly, in the diabetic rats that were consuming erythritol (DE group), the endothelium remained intact and the response to carbachol was identical to that observed in the normoglycemic rats. Reflecting this uncompromised response to carbachol is the smaller SNP contribution, similar to the contribution of SNP in the relaxation of aortic rings from normoglycemic rats, to the total relaxation of aortic segments in the erythritol supplemented diabetic rats.

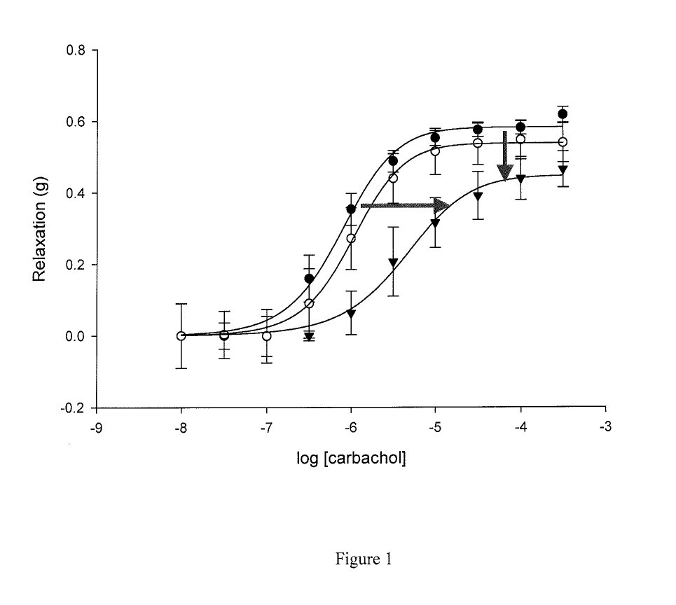

Furthermore, the carbachol concentration-response curves of diabetic rats (D), normoglycemic rats (N), and normoglycemic rats supplemented with erythritol (NE) are shown in FIG. 1. As seen in FIG. 1, the consumption of erythritol did not have a significant effect on the concentration-response curve in the normoglycemic rats. In contrast, the carbachol-response curve of aortic rings from diabetic rats (D) differed markedly from the curves obtained with rings from the normoglycemic rats (N) and from the erythritol-supplemented normoglycemic rats (NE) in that the curve was shifted to the right and the total relaxation was lowered.

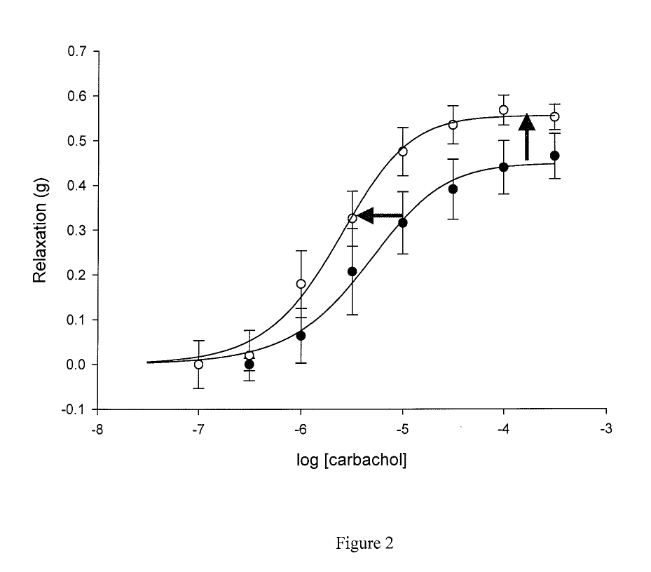

In FIG. 2, the carbachol concentration-response curves from diabetic rats (D) and erythritol-consuming diabetic rats (DE) are presented. The response to carbachol in the aortic rings from the rats of group DE was higher and at the same level as found in the aortic rings of the rats of groups N and NE. These results indicate that erythritol is able to prevent development of endothelial dysfunction in the diabetic rat and protect against hypertension.

### Example 2

In this second example, the present invention demonstrates the use of erythritol for the delaying and reducing hemolysis of red blood cells in an in vitro experiment. By delaying and reducing hemolysis of red blood cells, hemolysis-induced hypertension can be prevented, reduced or treated.

### Experimental Procedures

The effect of erythritol on 2,2′-azobis-2-amidinopropane dihydrochloride (AAPH)-induced hemolysis was investigated according to Vosters & Néve. A red blood cell suspension was zo incubated with erythritol (0-50 mM) for 5 min at 37° C. after which AAPH (50 mM) was added. At regular time intervals (0-300 min), 200 μl aliquots were taken and diluted in 2 ml 0.9% NaCl solution. The extent of hemolysis was determined by treating the sample with Hemoglobina TC® (potassium hexocyanoferrate (III) 2.4 mM, potassium cyanide 3 mM) for haemoglobin determination. The absorbance of the mixture was measured at 540 nm. 100% hemolysis was obtained by adding a 200 μl aliquot to 2 ml demineralized water. The percentage of hemolysis was calculated from the ratio (hemolysis in the test sample)-(100% hemolysis). The time needed to obtain 50% hemolysis (t50) was determined for each concentration by fitting the data with the 3-parameter sigmoid model from SigmaPlot 2001 for Windows (SPSS Inc., Chicago, USA).

### Results

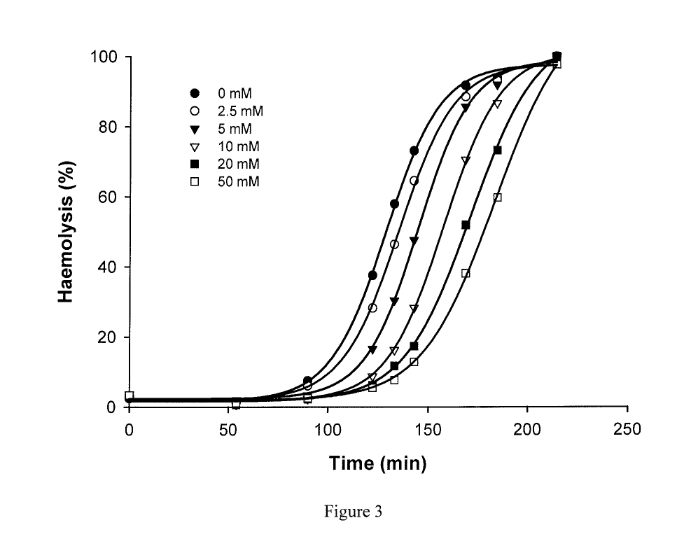

The effect of erythritol (0-50 mM) on AAPH-induced hemolysis is shown in FIG. 3. Erythritol caused a concentration-dependent increase in the lagtime (the time it takes before hemolysis starts). The lagtime was quantified by determining the t50, or the time it takes to reach 50% hemolysis. Erythritol inhibited hemolysis in a concentration-dependent manner, thereby demonstrating membrane-protective properties.

### Example 3

### Manganese Superoxide Dismutase Expression

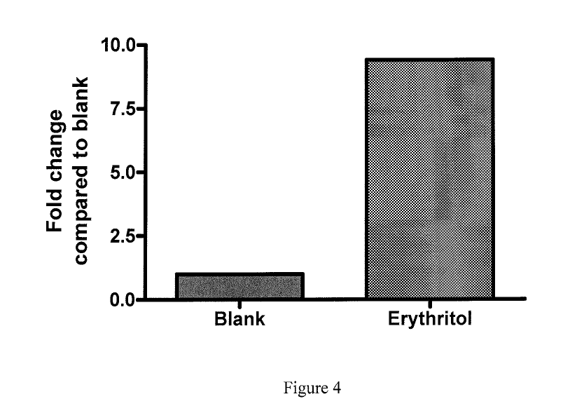

The exposure of endothelial cells to erythritol results in a increase in manganese superoxide dismutase (SOD2) transcription (FIG. 4). Cultured human umbilical vein endothelial cells (CRL-1730, which are available from ATCC) were incubated under standard conditions with erythritol (5 mM, 24 hours). Exposure to erythritol induced or stimulated a 9.4 fold increase in the expression of SOD2 as determined by qPCR (FIG. 4).

### Gene Expression Analysis

After incubation, RNA was isolated from Qiazol suspended cells according to the manufacturer's protocol and quantified using a nanodrop. Reverse transcription reaction was performed using 500 ng of total RNA, which was reverse-transcribed into cDNA using iScript™ cDNA synthesis kit. Next, real time PCR was performed with a BioRad MyiQ iCycler Single Color RT-PCR detection system using Sensimix™ Plus SYBR and Fluorescein, 5 μl diluted (10×) cDNA, and 0.3 μM primers in a total volume of 25 μl. PCR was conducted as follows: denaturation at 95° C. for 3 minutes, followed by 40 cycles of 95° C. for 15 seconds and 60° C. for 45 seconds. After PCR a melt curve (60-95° C.) was produced for product identification and purity. β-actin was included as internal control. Primer sequences are shown in table 2. Data were analysed using the MyIQ software system (Biorad) and were expressed as relative gene expression (fold change) using the 2^{ΔΔCt} method.

TABLE 2
Primer sequences for genes used for
gene expression analysis

| Gene name | Forward (5′ to 3′) | Reverse (5′ to 3′) |
|---|---|---|
| β-actin | CCTGGCACCCAGCACAAT | GCCGATCCACACGGAGTACT |
| SOD2 | ATCAGGATCCACTGCAAGGAA | CGTGCTCCCACACATCAATC |

1-10. (canceled)

11. A method of using erythritol for the manufacture of a product or composition for stimulation of SOD2 expression in an animal or human.

12. A method of using erythritol for the manufacture of a product or composition for the prevention or treatment of endothelial dysfunction or hemolysis in an animal or human.

13. A method of using erythritol for the manufacture of a product or composition for the prevention or treatment of hypertension in an animal or human.

14. The method of claim 3, wherein the hypertension that is prevented or treated is endothelial dysfunction-induced hypertension.

15. The method of claim 3, wherein the hypertension that is prevented or treated is hemolysis-induced hypertension.

16. The method of claim 2, wherein endothelial dysfunction is associated with hyperglycemia, diabetes, metabolic syndrome, cardiovascular disease, obesity, atherosclerosis, or inflammation.

17. The method of claim 2, wherein the hemolysis is associated with coronary artery disease, chronic heart failure, peripheral artery disease, atherosclerosis, diabetes, metabolic syndrome, obesity, inflammation, and chronic renal failure.

18. The method of claim 1 wherein the product or composition is orally administered.

19. The method of claim 3 wherein the product or composition is orally administered.

20. The method of claim 8, wherein the product or composition is a beverage or food.

21. The method of claim 9, wherein the product or composition is a beverage or food.

22. The method of claim 9, wherein the product or composition is a pharmaceutical.
